(19) United States
(12) Patent Application Publication
(10) Pub. No.: US 2023/0171562 A1
(43) Pub. Date: Jun. 1, 2023

(54) SYSTEMS AND METHODS FOR TRACKING DEVICES

(71) Applicant: HAPPY HEALTH INC., Austin, TX (US)

(72) Inventor: David E. CLIFT-REAVES, Austin, TX (US)

(73) Assignee: HAPPY HEALTH INC., Austin, TX (US)

(21) Appl. No.: 17/922,061

(22) PCT Filed: Apr. 28, 2021
(86) PCT No.: PCT/US2021/029537

§ 371 Date: Oct. 28, 2022

Related U.S. Application Data
(60) Provisional application No. 63/016,817, filed on Apr. 28, 2020.

(51) Int. Cl. H04W 4/029; H04W 4/02; G01S 1/04

(52) U.S. Cl. CPC H04W 4/029; H04W 4/023; G01S 1/042; G06Q 50/01

(57) ABSTRACT

A method is provided to receive, by a first device, a first beacon generated by a second device. The first beacon includes a first unique identifier. Based on the first beacon, the first device is triggered to transmit a second beacon including a second unique identifier. It can be determined that the first device and the second device are within a predetermined distance based on the first beacon and the second beacon. The first device receives a first set of data that includes the location information of the second device. The first set of data is decoded based on the first unique identifier.

### CROSS-REFERENCE TO RELATED APPLICATION

This application claims priority benefit to U.S. Provisional Patent Application No. 63/016,817, filed in the U.S. Patent and Trademark Office on Apr. 28, 2020, all of which is incorporated herein by reference in its entirety for all purposes.

### FIELD

The present disclosure relates generally to systems and methods to track devices. In at least one example, the present disclosure relates to determining and tracking whether devices were within threshold distances from one another.

### BACKGROUND

Information regarding whether people cross paths can be utilized for a multitude of purposes. For example, the information can be utilized for contact tracing to help stem the spread of disease. In some examples, the information can be utilized for informing people that they have passed by their friends or persons of interest.

### BRIEF DESCRIPTION OF THE DRAWINGS

Implementations of the present technology will now be described, by way of example only, with reference to the attached figures, wherein:

FIG. 1 is a schematic view of an exemplary system for tracking devices;

FIG. 2 is a schematic diagram of an exemplary device according to the disclosure herein;

FIG. 3 illustrates a user within a restricted zone as determined by a global positioning system (GPS) and a user outside of the restricted zone;

FIG. 4A illustrates two users outside of a predetermined distance from one another;

FIG. 4B illustrates the two users being within a predetermined distance from one another;

FIG. 4C illustrates one user within a predetermined distance but the other user being outside of a predetermined distance;

FIG. 5 is a flow chart of a method for tracking devices according to the disclosure herein.

### DETAILED DESCRIPTION

It will be appreciated that for simplicity and clarity of illustration, where appropriate, reference numerals have been repeated among the different figures to indicate corresponding or analogous elements. In addition, while specific details are set forth in order to provide an understanding of the examples described herein, it will be appreciated by those skilled in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “communicatively coupled” is defined as connected, either directly or indirectly through intervening components, and the connections are not necessarily limited to physical connections, but are connections that accommodate the transfer of data between the so-described components. The connection can be such that the objects are permanently connected or releasably connected. The terms “comprising,” “including” and “having” are used interchangeably in this disclosure. The terms “comprising,” “including” and “having” mean to include, but not necessarily be limited to the things so described.

Where components are described as being “configured to” or “operable to” perform certain operations, such configuration or operation can be accomplished, for example, by designing electronic circuits or other hardware to perform the operation, by programming programmable electronic circuits (e.g., microprocessors, or other suitable electronic circuits) to perform the operation, or any combination thereof.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods, algorithms, and/or operations described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

The present disclosure provides for systems, methods, and computer-readable media for anonymously obtaining distance information for different devices. The disclosed technologies can provide accurate, secure and scalable tracing of users and devices. In some examples, the disclosed technologies can anonymously obtain distance information between devices. The distance information can be used to track and/or trace users accurately, efficiently, and anonymously. In at least one example, by being able to track and/or trace users anonymously and accurately, notifications can be sent to users to inform the users that they have passed by or come within a predetermined distance from one another. For example, such information can inform the user that they may have been exposed to disease. Also for example, such information can inform the user that they have been within a predetermined distance to socialize with someone in their contact list.

In some cases, the disclosed technologies can use any combination of global positioning system (GPS) data, Bluetooth low energy (BLE) beacons, and audio data to anonymously identify distance information between devices, and securely and anonymously track and/or trace users and devices in scale.

In some examples, GPS data from devices can be used to provide lower resolution location information and/or context information for key generation, database (DB) filtering, etc. For example, the GPS data can be utilized to determine whether the device is within a restricted zone. If the device is in the restricted zone, the device may stop tracking the location.

BLE beacons can be used by devices to communicate device global unique identifiers (GUIDs) and/or provide information about a physical distance/location between devices. For example, the beacons can be used to determine whether the devices are within a predetermined distance from one another.

Audio can also be used as a secondary source of physical distance information between devices. For example, when the devices are within the predetermined distance from one another, audio can be emitted to provide more accurate data on physical distance/location between devices.

The information from the GPS data, BLE beacons, and/or audio can be combined to generate anonymous, higher-resolution distance information for devices. The GPS data, BLE beacons, and/or audio can also be used to trigger certain actions and/or functionalities to facilitate the generation of distance information. In some examples, other types of sensors or communications devices can also be used to calculate the distance information. For example, accelerometers, gyroscopes, inertial measurement units (IMUs), radars, image sensors, etc., can be used to calculate motion information, distance information, trajectories, proximities to surrounding devices/objects, etc., and combined with the GPS data, BLE beacons, and/or the audio to determine distance information for devices.

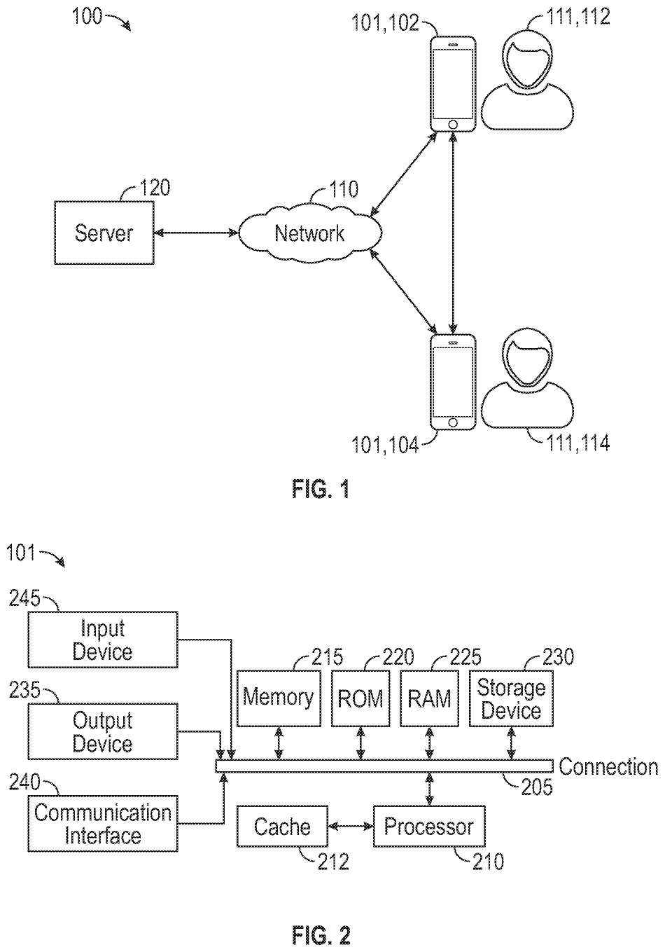

FIG. 1 is a block diagram illustrating an example system 100 for anonymously obtaining distance information between devices, in accordance with some examples of the present disclosure. In this example, the system 100 includes a plurality of devices 101 that are associated with users 111, a network 110, and a server 120. For example, as illustrated in FIG. 1, the device 102 is associated with user 112, and the device 104 is associated with user 114.

The devices 101 can include any mobile computing device with BLE capabilities and audio capabilities (e.g., speaker capabilities, microphone capabilities, etc.). In some examples, the devices 101 can have GPS capabilities. Non-limiting examples of devices 101 can include a smartphone, a smart wearable device (e.g., a smart watch, smart ring, a head-mounted display (HMD), smart glasses, etc.), a tablet computer, a laptop computer, a mobile computing device, etc. In some examples, any combination of devices 101 can work together to perform any of the disclosed functions. For example, a smart ring may be paired with a smart phone. The smart phone may perform a number of functions such as broadcasting a beacon while the smart ring may perform a number of functions such as receiving audio data. The data from the smart phone and the smart ring may be consolidated in the smart phone, the smart ring, and/or a server to be analyzed.

Network 110 can represent one or more public and/or private networks. For example, network 110 can include a wireless network (e.g., a cellular network, a wireless local area network, a satellite communication network, a microwave network, etc.), an IP network, a public cloud network, a private cloud network, a hybrid or multi-cloud network, a virtual network, a local area network, the Internet, a wide area network, and/or any other type of network.

The server 120 can include one or more servers, server applications, cloud services, datacenters, databases, computing systems, etc. The server 120 can communicate with the devices 101 via the network 110. In some examples, the devices 101 can also communicate with each other and/or with other devices via the network 110.

The devices 101 can communicate wirelessly with each other. For example, the devices 101 can communicate with each other via a point-to-point or direct wireless connection such as, for example, a Bluetooth connection, a WIFI-direct connection, etc. In some cases, the devices 101 can communicate with each other via a wireless local area network such as, for example, a WIFI network.

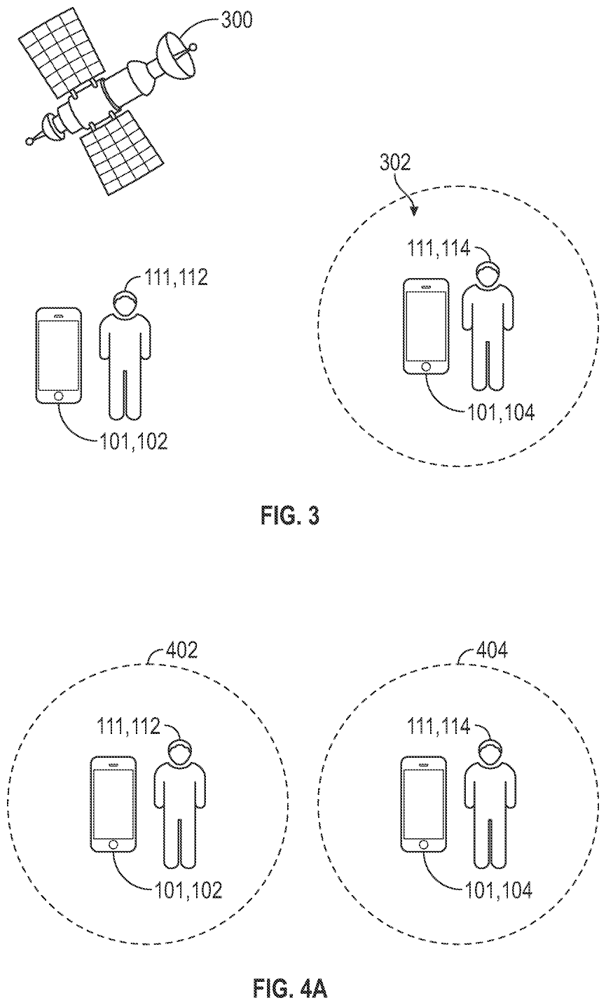

FIG. 2 illustrates an example computing device architecture of an example device 101 which can implement various techniques described herein. The components of the computing device 101 are shown in electrical communication with each other using a connection 205, such as a bus. The example device 101 includes a processing unit (CPU or processor) 210 and a computing device connection 205 that couples various computing device components including the computing device memory 215, such as read only memory (ROM) 220 and random access memory (RAM) 225, to the processor 210.

The device 101 can include a cache of high-speed memory connected directly with, in close proximity to, or integrated as part of the processor 210. The device 101 can copy data from the memory 215 and/or the storage device 230 to the cache 212 for quick access by the processor 210. In this way, the cache can provide a performance boost that avoids processor 210 delays while waiting for data. These and other modules can control or be configured to control the processor 210 to perform various actions. Other computing device memory 215 may be available for use as well. The memory 215 can include multiple different types of memory with different performance characteristics. The processor 210 can include any general purpose processor and a hardware or software service stored in storage device 230 and configured to control the processor 210 as well as a special-purpose processor where software instructions are incorporated into the processor design. The processor 210 may be a self-contained system, containing multiple cores or processors, a bus, memory controller, cache, etc. A multi-core processor may be symmetric or asymmetric.

To enable user interaction with the device 101, an input device 245 can represent any number of input mechanisms, such as a microphone for speech, a touch-sensitive screen for gesture or graphical input, keyboard, mouse, motion input, speech and so forth. An output device 235 can also be one or more of a number of output mechanisms known to those of skill in the art, such as a display, projector, television, speaker device. In some instances, multimodal computing devices can enable a user to provide multiple types of input to communicate with the device 101. The communication interface 240 can generally govern and manage the user input and computing device output. There is no restriction on operating on any particular hardware arrangement and therefore the basic features here may easily be substituted for improved hardware or firmware arrangements as they are developed.

Storage device 230 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 225, read only memory (ROM) 220, and hybrids thereof. The storage device 230 can include software, code, firmware, etc., for controlling the processor 210. Other hardware or software modules are contemplated. The storage device 230 can be connected to the computing device connection 205. In one aspect, a hardware module that performs a particular function can include the software component stored in a computer-readable medium in connection with the necessary hardware components, such as the processor 210, connection 205, output device 235, and so forth, to carry out the function.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some examples, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual embodiments may be described above as a process or method depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

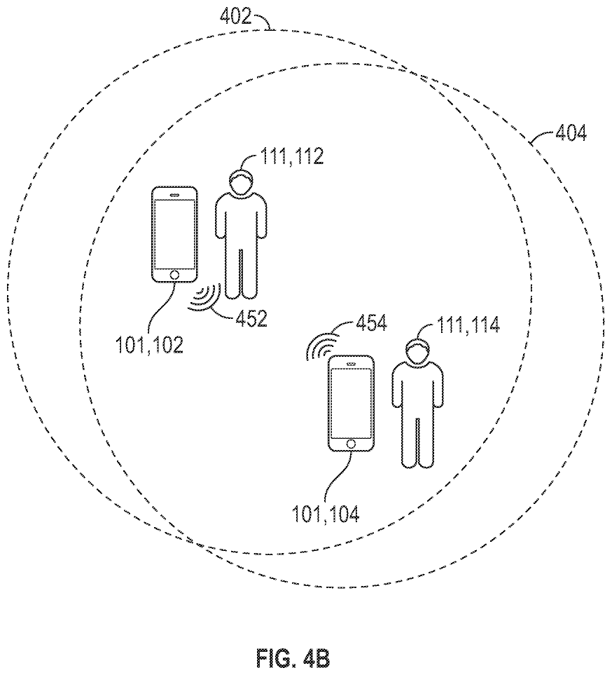

In some examples, as illustrated in FIG. 3, the GPS data from a device 101 can provide a path of the 111 associated with that device 101. For example, the device 101 may be in communication with a GPS satellite 300 which provides GPS data for the device 101. In some cases, the GPS for the device 101 can be set to a predetermined resolution. For example, the GPS can be set to a low resolution such as, for example, 1 kM. In some cases, the device can also obtain motion detection using accelerometer data. In some examples, GPS data can be used to provide a context and/or indicate world region information.

As illustrated in FIG. 3, the GPS data can be used to determine whether the device 101, for example device 104, is within a restricted zone 302. In at least one example, the functionality of the devices 101 to perform the tracking and/or tracing (e.g., BLE beacons and/or audio components) may be disabled when within the restricted zone 302. The restricted zone 302 can be any predetermined area where the devices 101 cannot be tracked and/or traced. For example, it may be desired that devices 101 and users 111 are not tracking and/or tracing other devices 101 and users 111 within a government building. The restricted zone 302 can include, for example, buildings, rooms, properties, and/or any other desired area to prevent tracking and/or tracing of devices 101 and corresponding users 111.

When the device 101 is not within the restricted zone 302, for example device 102 and user 112, the device 101 can function as normal in performing anonymous tracking and/or tracing with other devices 101 in the surrounding areas.

In some examples, the GPS data can be utilized to determine whether to activate the tracking and/or tracing functionalities of the devices 101. Accordingly, if the GPS data informs that the device 101 is within a predetermined zone, the tracking and/or tracing functionalities may be activated. For example, when a device 101 enters an activation zone such as festival grounds, the tracking and/or tracing functionalities may be activated such that the device 101 and user 111 can be notified when within a predetermined distance from devices 101 from a contact list. The user 111 can then more easily find other users 111 that they know and/or want to socialize with.

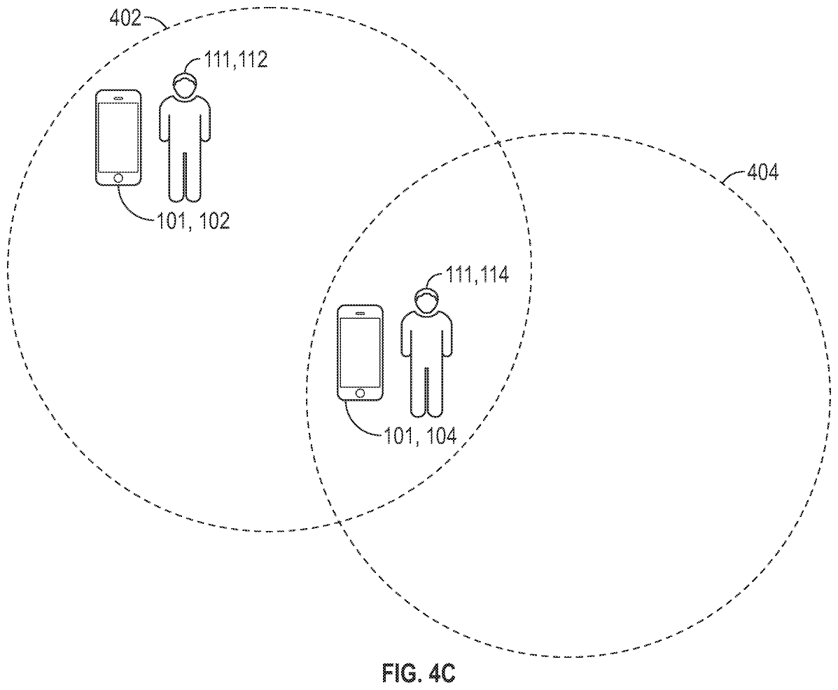

The device 101 can broadcast a beacon to locate and/or track other devices 101. In at least one example, the beacon can include BLE beacons. BLE beacons can transmit data through Bluetooth low energy by broadcasting the device's 101 unique identifier. The BLE beacon may be broadcasted out from the device 101 to have a spherical range. Other devices 101 may receive the BLE beacon when within the range. The device 101 can determine whether the other device 101 is within predetermined distances within the range of the broadcast. As illustrated in FIG. 4A, the device 102 has a beacon range 402, and the device 104 has a beacon range 404. As shown in FIG. 4A, the device 102 and the device 104 are not within the corresponding beacon ranges 402, 404. Accordingly, although device 102 and device 104 may be broadcasting the corresponding beacons, neither device 102 nor device 104 are receiving the beacon.

In at least one example, the beacon can be a one way transmitter to the receiving device 101, and may require a specific application on the receiving device 101 to interact with the beacon. In such example(s), only the installed application can be used to track the devices. For example, the application on device 102 can run in the background. Device 104 may or may not have an application running in the background. The application on the device 102 can use GPS to store location information associated with device 101 and/or set a BLE beacon to be transmitted from the device 101 in a certain way.

In at least one example, as illustrated in FIG. 4B, when the device 104 is within the beacon range 402 of device 102, device 104 may receive the beacon broadcast by the device 102. The BLE beacon from the device 102 can wake up an application on the device 104 and the application on the device 104 can start sending its own BLE beacon. Similarly, in some examples, when the device 102 is within the beacon range 404 of device 104, the device 102 may receive the beacon broadcast by the device 104.

When the device 104 is within a predetermined distance from the device 102, the device 102 may send a set of data 452 to the device 104. The set of data 452 can include the location information of the device 102. In some examples, when the device 102 is within a predetermined distance from the device 104, the device 104 may send a set of data 454 which can include the location information of the device 104. In some examples, the devices 101 can determine that the other device(s) 101 are within the predetermined distance based on the received beacons. In some examples, the server 120 can determine that the devices 101 are within the predetermined distance and instruct the devices 101 to send the sets of data 452, 454. With the sets of data 452, 454, it can be determined whether the devices 102, 104 are within a threshold distance from one another. The threshold distance can be a distance where interaction between the devices 102, 104 are possible. For example, within the threshold distance, disease may be spread. In some examples, within the threshold distance, the users 111 may be able to find and speak with one another. The threshold distance can be set and determined through the server and/or by each device 101.

In at least one example, the set(s) of data 452, 454 from the devices 102, 104 may be sent through audio data after receiving the beacon from the other device 102, 104 and determining that the other device 102, 104 is within a predetermined distance. For example, the device 104 can check its audio route (e.g., interface, path, port, etc.) for a speaker on the device 104. If the speaker on the device 104 is not being used (e.g., is not active, activated, enabled, turned on, etc.), then the device 104 can turn on an ultrasonic repeating beacon. The device 104 can also turn on a microphone if the microphone is not in use. The device 104 can use the GUID of the device 102, which it can acquire via the BLE beacon from the device 102, to configure a spread spectrum receiver function to process audio acquired from the device 102 via a microphone on the device 104, and continually store the amplitude of the audio signal that includes the set of data.

In some examples, the device 102 can receive a BLE response beacon from device 104 and turn on a microphone and speaker on the device 102. The device 102 can emit the set of data 452 through audio data, for example a spread spectrum signal, and look for a signal or response from device 104 to the spread spectrum signal.

In at least one example, when the BLE beacons disappear (for example are not received by the devices 101), then the devices 101 can both turn off their audio. In some examples, as illustrated in FIG. 4C, when the devices 102, 104 are not within the predetermined distance from one another but may still be within the beacon ranges 402, 404, the devices 102, 104 can stop emitting audio and stop sending the set(s) of data.

The information from the GPS data, the BLE beacons, and/or the audio signals can be used to determine a location and relative distances of the devices 101. This information can be used to accurately track the location of the devices 101 and the associated users 111. In some examples, the devices 101 can communicate the location information to the server 120.

In addition, the process described herein can maintain security and anonymity. For example, in some cases, neither device 102 nor device 104 may know who they were near and/or the identities of users 112 and 114. In some cases, only the server 120 may know which of the devices 101 were within a threshold distance. In some cases, when the devices 101 synchronize their local databases to the server 120, the server 120 can create a set of identified users 111 (e.g., 112, 114) seen for each user.

Various other layers of security can also be implemented. For example, the system 100 can allow each device 101 (and/or user 111) know that they have seen each the other or been near each other in order to ensure that other users or devices cannot just query a large number of users that they never saw or were near. In at least one example, a notification may be produced by the device 101 to inform the corresponding user 111 that another device was nearby within a threshold distance. This can inform the user 111, for example, that they were within a threshold distance to another user 111 and may have been exposed to a disease. In some examples, a social interaction probability score may be determined based on the location information of the devices 101. The social interaction probability score can be determined based on, for example, rate of travel, distance from the other device, and/or path of travel. When the social interaction probability score is above a social threshold, a notification may be provided to the device 101 to inform the user of the device 101 nearby within the predetermined distance. In some examples, the social interaction probability score may be determined by the server 120. The server 120 can then be operable to provide the notification to the devices 101. In some examples, the social interaction probability score may be determined by each individual device 101.

In some examples, the information in databases to the server 120 can be limited for security reasons. For example, a database can include device's 101 unique IDs used, timing information (e.g., when), location information (e.g., where), etc. Other example information can include a start time, a start coordinate, a stop time, a stop coordinate, a local device previous GUID, a local device current GUID, etc.

In some examples, the system can include a database of GUIDs seen via BLE beacons. In some examples, the information can include time, coordinate, local device GUID, remote device GUID, RSSI (received signal strength indicator), etc.

In some examples, the system can include a database of GUIDs heard via audio. In some examples, the information can include time, coordinate, local device GUID, remote device GUID, sound amplitude, etc.

In some examples, the BLE beacon can be configured with a certain format and/or information associated with a device. For example, a BLE beacon can include a GUID associated with a device and/or an application on the device. The BLE beacon can include a major and minor, such as 2 bytes for the major and 2 bytes for the minor. In some cases, the GPS region (e.g., identified based on the GPS data) can set the BLE beacon major or can be embedded in the application or device GUID. In some examples, part of the GUID can be used to encode the GPS region which, in some cases, can make knowing which beacons to listen for trivial on the device side and/or may not require a server hit or look up table.

In some examples, the first two bytes of a GUID can set can be static and the last 2 bytes can include 1 byte worth of latitude information and 1 byte of longitude information. This example can provide roughly 100×100 mile blocks.

In some examples, 4 bytes for major/minor can be used for a user GUID. This GUID can change when the user changes geographic regions or after a period of time. The users IDs, time started/stopped, and/or GPS coordinates can be stored in a local DB. Periodically, this information can be sent to the server 120.

As previously noted, the BLE beacon transmitted from one device can be used to turn on the application on the other device via a low power method. The application on the other device can be triggered by the BLE beacon to start broadcasting and listening via an audio interface.

In some examples, audio adaptations/techniques can be implemented to provide finer distance resolution. For example, techniques can be implemented to give finer resolution on distance from another device. In some cases, the security can be maintained by a third partly listener who does not know the application's protocol.

In some examples, the BLE beacons can be used as an OOB (out of band) communication channel for reporting a GUID. Moreover, a function can be used to map GUIDs to a spread spectrum pattern that will correspond to or be the audio output. To a normal or unintended listener, this can appear as random noise. However, since the intended listener/recipient device (e.g., device and/or server) knows the GUID, the intended listener/recipient can decode the audio information. Thus, this strategy can provide a level of audio encryption or obfuscation.

In some examples, the output from this process can be a single dimension output. In some examples, an amplitude of the audio code can be detected. This can be used to estimate device distance information. In some cases, this can be used with some level of normalization to estimate device distance.

In some examples, the audio processing can be processor efficient. However, in some examples, leaving the hardware for a speaker/microphone on at all times can be expensive even if no processing is occurring. Accordingly, in some examples, the speaker and/or microphone may only be turned on if there is knowledge or an indication that another device is near and within a predetermined distance. In some examples, there can also be a shared idea of when a duty cycle should be.

In some examples, the spread spectrum can sound like noise and/or can be a low amplitude over a large spectrum so the entire audio band can be used and to avoid notifying the user or device of the broadcasting.

This method can allow numerous devices to be broadcasting loudly next to one another and each receiver to only hear the broadcasters(s) they are listening for.

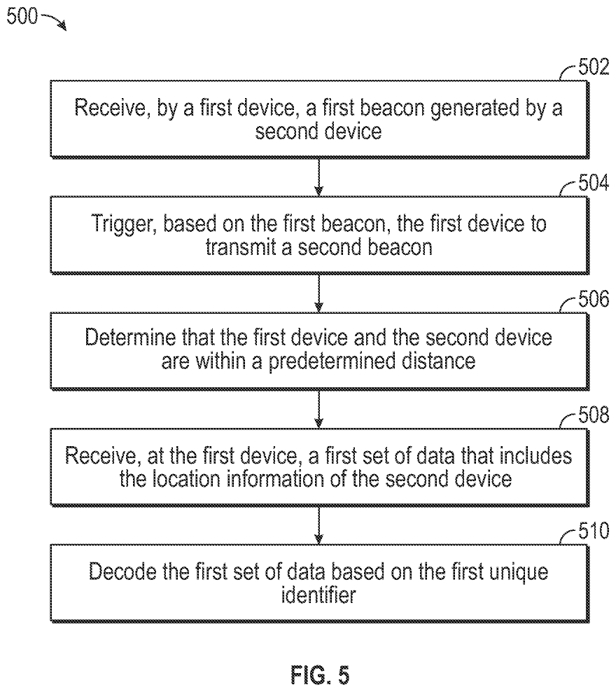

FIG. 5 is an example method 500 for tracking and/or tracing devices, in accordance with various aspects of the subject technology. The method 500 is provided by way of example, as there are a variety of ways to carry out the method. The method 600 described below can be carried out using the configurations illustrated in FIGS. 1-4C, for example, and various elements of these figures are referenced in explaining example method 500. Each block shown in FIG. 5 represents one or more processes, methods or subroutines, carried out in the example method 500. Furthermore, the illustrated order of blocks is illustrative only and the order of the blocks can change according to the present disclosure. Additional blocks may be added or fewer blocks may be utilized, without departing from this disclosure. The method 500 can begin at block 502.

At block 502, a first device receives a first beacon generated by a second device. The first beacon can include a first unique identifier. In at least one example, the first beacon can include a BLE beacon. At block 504, the first device is triggered, based on the first beacon, to transmit a second beacon including a second unique identifier. In at least one example, the first beacon and/or the second beacon include a Bluetooth low energy beacon.

In at least one example, based on the GPS data of the first device, it can be determined whether the first device is within a restricted zone. The determination can be performed by the server and/or the first device. When the first device is in the restricted zone, the first device can stop transmitting the second beacon. Similarly, GPS data of the second device can indicate whether the second device is in a restricted zone. When the second device is in the restricted zone, the second device can stop transmitting the first beacon.

At block 506, it is determined that the first device and the second device are within a predetermined distance based on the first beacon and the second beacon. This determination can be performed by the server, the first device, and/or the second device.

At block 508, the first device receives a first set of data that includes the location information of the second device. The second device can emit the first set of data upon determination that the first device and the second device are within the predetermined distance based on the first beacon and the second beacon. In at least one example, upon determining that the first device and the second device are within the predetermined distance, the first device can emit a second set of data that includes the location information of the first device.

In at least one example, the first and/or second set of data can be included in audio data. The audio data can be received through an audio channel and/or a radiofrequency channel. The audio data can include a spread spectrum signal.

At block 510, the first set of data is decoded based on the first unique identifier. In at least one example, the first device can decode the first data. In some examples, the server can decode the first set of data after receiving the first set of data from the first device. In at least one example, the first device can decode the first set of data by configuring a spread spectrum receiver to process the audio data and store and amplitude of audio.

After determining the location information of the second device, it can be determined whether the first device passed within a threshold distance from the second device based on the location information of the first device and the location information of the second device. The threshold distance can be determined based on the first set of data and/or the second set of data. In at least one example, the threshold distance can be determined by the server. In some examples, the threshold distance can be determined by the first device and/or the second device. If the first device and the second device passed within the threshold distance, in at least one example, a notification can be provided by the first device and/or the second device to inform the user. For example, the notification can inform the user(s) of the first device and/or the second device that they were exposed to a disease after passing within the threshold distance from another user.

In at least one example, a social interaction probability score can be generated based on the location information of the first device and the location information of the second device. The social interaction probability score can be determined based on, for example, rate of travel, distance from the other device, and/or path of travel. When the social interaction probability score is above a social threshold, a notification may be provided to the first device and/or the second device to inform the user(s) of the device(s) nearby within the predetermined distance. In some examples, the social interaction probability score may be determined by the server. The server can then be operable to provide the notification to the devices. In some examples, the social interaction probability score may be determined by each individual device.

In at least one example, when the first device does not receive the first beacon, it may be determined that the first device is within the predetermined distance from the second device. Upon such determination, the first device may stop transmitting the second beacon. In some example, the first device may continue transmitting the second beacon and only stop transmitting the second set of data. Similarly, the second device may stop transmitting the first beacon and/or the first set of data.

In the foregoing description, aspects of the application are described with reference to specific embodiments thereof, but those skilled in the art will recognize that the application is not limited thereto. Thus, while illustrative embodiments of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, embodiments can be utilized in any number of environments and applications beyond those described herein without departing from the broader spirit and scope of the specification. The specification and drawings are, accordingly, to be regarded as illustrative rather than restrictive. For the purposes of illustration, methods were described in a particular order. It should be appreciated that in alternate embodiments, the methods may be performed in a different order than that described.

Numerous examples are provided herein to enhance understanding of the present disclosure. A specific set of statements are provided as follows.

Statement 1: A method is disclosed comprising: receiving, by a first device, a first beacon generated by a second device, wherein the first beacon including a first unique identifier; triggering, based on the first beacon, the first device to transmit a second beacon including a second unique identifier; determining that the first device and the second device are within a predetermined distance based on the first beacon and the second beacon; receiving, at the first device, a first set of data that includes the location information of the second device; decoding the first set of data based on the first unique identifier.

Statement 2: A method is disclosed according to Statement 1, further comprising: determining, based on GPS data of the first device, whether the first device is within a restricted zone; when the first device is in the restricted zone, stop transmitting the second beacon.

Statement 3: A method is disclosed according to Statements 1 or 2, wherein the first beacon and/or the second beacon include a Bluetooth low energy beacon.

Statement 4: A method is disclosed according to any of preceding Statements 1-3, wherein the first set of data is included in audio data.

Statement 5: A method is disclosed according to Statement 4, wherein the audio data is received through an audio channel and/or a radiofrequency channel.

Statement 6: A method is disclosed according to Statements 4 or 5, wherein the audio data includes a spread spectrum signal.

Statement 7: A method is disclosed according to Statement 6, wherein the decoding the first set of data includes: configuring, by the first device, a spread spectrum receiver to process the audio data and store and amplitude of audio.

Statement 8: A method is disclosed according to any of preceding Statements 1-7, further comprising: emitting, by the first device upon determining that the first device and the second device are within the predetermined distance, a second set of data that includes the location information of the first device.

Statement 9: A method is disclosed according to Statement 8, wherein the second set of data is included in audio data and includes a spread spectrum signal.

Statement 10: A method is disclosed according to any of preceding Statements 1-9, further comprising: determining the location information of the second device; determining whether the first device passed within a threshold distance from the second device based on location information of the first device and the location information of the second device.

Statement 11: A method is disclosed according to Statement 10, further comprising: generating, based on the location information of the first device and the location information of the second device, a social interaction probability score; when the social interaction probability score is above a social threshold, provide a notification to the first device and/or the second device.

Statement 12: A method is disclosed according to any of preceding Statements 1-11, further comprising: determining, when the first device does not receive the first beacon, the first device is not within the predetermined distance from the second device; stop transmitting, by the first device, the second beacon.

Statement 13: A system is disclosed comprising: a first device; a second device operable to generate a first beacon, the first beacon including a first unique identifier; wherein upon receiving the first beacon, the first device transmits a second beacon including a second unique identifier; wherein when the second device is within a predetermined distance from the first device based on the first beacon and the second beacon, the first device is operable to receive a first set of data from the second device and decode the first set of data based on the first unique identifier.

Statement 14: A system is disclosed according to Statement 13, wherein when the first device based on GPS data is in a restricted zone, the first device is operable to stop broadcasting the second beacon.

Statement 15: A system is disclosed according to Statements 13 or 14, wherein the first beacon and/or the second beacon include a Bluetooth low energy beacon.

Statement 16: A system is disclosed according to any of preceding Statements 13-15, wherein the first set of data is included in audio data.

Statement 17: A system is disclosed according to Statement 16, wherein the audio data is received through an audio channel and/or a radiofrequency channel.

Statement 18: A system is disclosed according to Statements 16 or 17, wherein the audio data includes a spread spectrum signal.

Statement 19: A system is disclosed according to Statement 18, wherein the first device is operable to decode the first set of data by configuring a spread spectrum receiver to process the audio data and store an amplitude of audio.

Statement 20: A system is disclosed according to any of preceding Statements 13-19, wherein upon determining that the first device an the second device are within the predetermined distance, the first device is operable to emit a second set of data that includes the location information of the first device.

Statement 21: A system is disclosed according to Statement 20, wherein the second set of data is included in audio data and includes a spread spectrum signal.

Statement 22: A system is disclosed according to any of preceding Statements 13-21, further comprising: a server communicatively coupled with the first device and the second device, wherein the server is operable to determine the location information of the first device and the location information of the second device, wherein the server is operable to determine whether the first device passed within a threshold distance from the second device based on the location information of the first device and the location information of the second device.

Statement 23: A system is disclosed according to Statement 22, wherein the server is operable to generate, based on the location information of the first device and the location information of the second device, a social interaction probability score; wherein when the social interaction probability score is above a social threshold, the server is operable to provide a notification to the first device and/or the second device.

Statement 24: A system is disclosed according to any of preceding Statements 13-23, wherein, when the first device does not receive the first beacon, the first device stops transmitting the second beacon.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. A method comprising:
receiving, by a first device, a first beacon generated by a second device, wherein the first beacon including a first unique identifier;
triggering, based on the first beacon, the first device to transmit a second beacon including a second unique identifier;
determining that the first device and the second device are within a predetermined distance based on the first beacon and the second beacon;
receiving, at the first device, a first set of data that includes the location information of the second device;
decoding the first set of data based on the first unique identifier.

2. The method of claim 1, further comprising:
determining, based on GPS data of the first device, whether the first device is within a restricted zone;
when the first device is in the restricted zone, stop transmitting the second beacon.

3. The method of claim 1, wherein the first beacon and/or the second beacon include a Bluetooth low energy beacon.

4. The method of claim 1, wherein the first set of data is included in audio data.

5. The method of claim 4, wherein the audio data is received through an audio channel and/or a radiofrequency channel.

6. The method of claim 4, wherein the audio data includes a spread spectrum signal.

7. The method of claim 6, wherein the decoding the first set of data includes:
configuring, by the first device, a spread spectrum receiver to process the audio data and store an amplitude of audio.

8. The method of claim 1, further comprising:
emitting, by the first device upon determining that the first device and the second device are within the predetermined distance, a second set of data that includes the location information of the first device.

9. The method of claim 8, wherein the second set of data is included in audio data and includes a spread spectrum signal.

10. The method of claim 1, further comprising:
determining the location information of the second device;
determining whether the first device passed within a threshold distance from the second device based on location information of the first device and the location information of the second device.

11. The method of claim 10, further comprising:
generating, based on the location information of the first device and the location information of the second device, a social interaction probability score;
when the social interaction probability score is above a social threshold, provide a notification to the first device and/or the second device.

12. The method of claim 1, further comprising:
determining, when the first device does not receive the first beacon, the first device is not within the predetermined distance from the second device;
stop transmitting, by the first device, the second beacon.

13. A system comprising:
a first device;
a second device operable to generate a first beacon, the first beacon including a first unique identifier;
wherein upon receiving the first beacon, the first device transmits a second beacon including a second unique identifier;
wherein when the second device is within a predetermined distance from the first device based on the first beacon and the second beacon, the first device is operable to receive a first set of data from the second device and decode the first set of data based on the first unique identifier.

14. The system of claim 13, wherein when the first device based on GPS data is in a restricted zone, the first device is operable to stop broadcasting the second beacon.

15. The system of claim 13, wherein the first beacon and/or the second beacon include a Bluetooth low energy beacon.

16. The system of claim 13, wherein the first set of data is included in audio data.

17. The system of claim 16, wherein the audio data is received through an audio channel and/or a radiofrequency channel.

18. The system of claim 16, wherein the audio data includes a spread spectrum signal.

19. The system of claim 18, wherein the first device is operable to decode the first set of data by configuring a spread spectrum receiver to process the audio data and store an amplitude of audio.

20. The system of claim 13, wherein upon determining that the first device and the second device are within the predetermined distance, the first device is operable to emit a second set of data that includes the location information of the first device.

21. The system of claim 20, wherein the second set of data is included in audio data and includes a spread spectrum signal.

22. The system of claim 13, further comprising:
a server communicatively coupled with the first device and the second device,
wherein the server is operable to determine the location information of the first device and the location information of the second device,
wherein the server is operable to determine whether the first device passed within a threshold distance from the second device based on the location information of the first device and the location information of the second device.

23. The system of claim 22, wherein the server is operable to generate, based on the location information of the first device and the location information of the second device, a social interaction probability score; wherein when the social interaction probability score is above a social threshold, the server is operable to provide a notification to the first device and/or the second device.

24. The system of claim 13, wherein, when the first device does not receive the first beacon, the first device stops transmitting the second beacon.
